(19) United States
(12) Patent Application Publication
(10) Pub. No.: US 2007/0166698 A1
(43) Pub. Date: Jul. 19, 2007

(54) Assay Method for Drugs to Treat Atherosclerosis, Cancer and Alzheimer's Disease

(75) Inventor: Huntington Potter, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/686,631

(22) Filed: Mar. 15, 2007

Related U.S. Application Data
(63) Continuation of application No. PCT/US05/33522, filed on Sep. 19, 2005.
(60) Provisional application No. 60/522,343, filed on Sep. 17, 2004.

(51) Int. Cl. C12Q 1/60; C12Q 1/00

(52) U.S. Cl. 435/4; 435/11

(57) ABSTRACT

An assay to screen for potentially beneficial drugs against atherosclerosis, cancer and Alzheimer's disease, and their effectiveness in vitro. The assay is comprised of a system where LDL or cholesterol is given to various cells together with the drug being tested and then chromosome segregation and aneuploidy are detected using probes specific for targeted chromosomes. A related assay tests the ability of the drugs alter the fluidity of membranes.

### CROSS REFERENCE TO RELATED APPLICATIONS

This application is a continuation of International Application No.

PCT/US2005/033522 entitled, “Assay Method for Drugs to threat Atherosclerosis Cancer and Alzheimer's Disease”, filed Sep. 15, 2005, which claims the benefit of priority to U.S. Provisional Patent Application 60/522,343, entitled, “Screen for Drugs to Treat Atherosclerosis Cancer and Alzheimer's Disease”, filed Sep. 17, 2004, the contents of which are herein incorporated by reference.

### GOVERNMENT SUPPORT

This invention was developed under support from the National Institute for Health under grant AG09665; accordingly, the U.S. government may have certain rights in the invention.

### BACKGROUND OF THE INVENTION

In conditions with elevated concentrations of Low-Density-Lipoproteins (LDL particles), cholesterol promotes atheroma plaque deposits in the walls of arteries, a condition known as atherosclerosis, which is a major contributor to coronary heart disease and other forms of cardiovascular disease. Atherosclerosis is also associated with debilitating and deadly disease in millions of people world wide, as are cancer and Alzheimer's disease.

Aneuploidy is a chromosomal state where abnormal numbers of specific chromosomes or chromosome sets exist within the nucleus. Aneuploidy is highly prevelant in cancer cells suggesting that failure of the mitotic apparatus is a major step in the conversion of a normal cell into a cancerous one.

Therefore, what is needed is a screening system for drug candidates to treat these diseases by means of an assay system to look for compounds which reduce the presence of chromosome aneuploidy.

### SUMMARY OF INVENTION

In one embodiment, the present invention includes an assay to screen for potentially beneficial drugs, in vitro, against atherosclerosis, cancer and Alzheimer's disease. The assay is comprised of a system where LDL or cholesterol is given to various cells together with the drug being tested and then chromosome segregation and aneuploidy are detected using probes specific for targeted chromosomes, as is known in the art.

In an illustrative embodiment the method of identifying an agent capable of inhibiting chromosome missegregation comprises the steps of providing a test cell and administering to said test cell an experimentally effective amount of a solution comprising a compound such as cholesterol or low-density-lipoprotein (LDL). The test cell is then exposed to an agent suspected of inhibiting chromosome missegregation. The test cells are allowed to reproduce, thereby producing a population of progeny cells. By studying the population of progeny cells, the number of aneuploid cells in said population of progeny cells is indicative of the agents is effectiveness of inhibiting chromosome missegregation. It is beneficial that the test cell express telomerase reverse transcriptase.

Useful concentrations of low-density-lipoprotein solution for the present assay were found at about 20 μg/ml. Water soluble cholesterol, in concentrations between about 2 and 4 μg/ml are also employed. Standard metaphase chromosome analysis, or any system known in the art for detecting aneuploidy, is used to determine the number of anomalous cells in said population of progeny cells.

In an alternate embodiment, the present invention provides for a method of identifying an agent capable of modulating membrane fluidity comprising the steps of providing a test cell and administering to said test cell an experimentally effective amount of a solution comprising a compound such as cholesterol and/or low-density-lipoprotein. As with the previous embodiment, the test cells are exposed to an agent suspected of inhibiting chromosome missegregation. The test cells are allowed to reproduce, thereby producing a population of progeny cells. By studying the population of progeny cells, the number of aneuploid cells in said population of progeny cells is indicative of the agents is effectiveness of inhibiting chromosome missegregation. It is beneficial that the test cell express telomerase reverse transcriptase.

### BRIEF DESCRIPTION OF THE DRAWINGS

For a fuller understanding of the nature and objects of the invention, reference should be made to the following detailed description, taken in connection with the accompanying drawings, in which:

FIG. 1 is a graph showing the frequency of aneuploid cells resulting from 7 experiments, A through G, with Oxidized LDL (OX-LDL), LDL, HDL and human telomerase reverse transcriptase (HTERT).

FIG. 2 is a graph showing the average frequency of aneuploid cells resulting from the 7 experiments with Oxidized LDL (OX-LDL), LDL, HDL and human telomerase reverse transcriptase (HTERT).

FIG. 3 is a graph showing the average frequency of aneuploid cells resulting from 3 experiments with water soluble cholesterol.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

As disclosed herein, there is a correlation between serum LDL levels and plaque formation in atherosclerosis and in cancer and Alzheimer's disease. This invention serves to provide a screening assay to determine the effect of therapeutic drugs on chromosome segregation leading to aneuploidy, which underlies these and other age-related disorders. The inventors have established a direct correlation between LDL exposure and chromosome missegregation, with HDL having little or no effect. Addition of cholesterol made water soluble, as by cyclodextrin, also increases chromosome missegregation. The future use of the novel screening assay is to look for therapeutic compounds which will prevent the development of chromosome aneuploidy in LDL or cholesterol treated cells. The second embodiment of the invention will screen compounds for their effect on lipid membrane fluidity, which cholesterol and LDL also affect and may be the means by which they cause chromosome missegregation. A putative atherosclerotic compound drug identified by either assay can then be tested to determine if it prevents the formation of atherosclerotic plaques in vivo. The potential use of these assays will lead the way for potential drug candidates to be generated to treat atherosclerosis, cancer and Alzheimer's disease.

Assay

The invention provides a method (also referred to herein as a “screening assay”) for identifying modulators, i.e., candidate or test compounds or agents (e.g., peptides, peptidomimetics, small molecules or other drugs) which have a stimulatory or inhibitory effect on chromosome missegregation, resulting in aneuploidy. Such identified compounds may be useful for the treatment of diseases associated with plaque formation such as Alzheimer's disease, cancer, and atherosclerosis. The test compounds of the present invention can be presented using any of the numerous approaches in combinatorial library methods known in the art, including: biological libraries; natural products libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909;

Erb et al. (1994) Proc. Natl. Acad. Sci. USA 91:11422; Zuckermann et al. (1994). J.

Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed.

Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol: Biol. 222:301-310).

In an illustrative embodiment, the assay is a cell-based assay in which a test cell expressing telomerase reverse transcriptase is contacted with a test compound and a solution comprising low-density-lipoprotein (LDL) or cholesterol. The ability of the test compound to inhibit missegregation is then determined by assessing the number of anomalous cells in the progeny of the test cell. The test cell is preferably a cell of mammalian, ideally human, origin.

The term “modulating” as used herein means changing, adjusting, or varying a property of a tissue, cell, or molecule. For example, modulation may cause an increase or a decrease in membrane rigidity, cellular activity, growth, inhibited DNA synthesis, apoptosis or any other biological, functional or immunological properties associated with the assay.

An “experimentally effective amount” of solution is that amount necessary to induce chromosome missgegration. The amount of solution must be effective to achieve a response, including but not limited to chromosome missegregation, increased membrane rigidity, aneuplooidy, or other indicators as are selected as appropriate measures by those skilled in the art.

Methods

hTERT-HME1 cell line

The hTERT-HME1 cell line, a primary human mammary epithelial cell line that permanently expresses the telomerase reverse transcriptase, was used as the test cell line. Stable expression of telomerase reverse transciptase maintains telomere length and allows the cells to divide indefinitely while retaining normal function, phenotype, and karyotype (Jiang et al., 1999; Morales et al., 1999).

The hTERT-HME1 cell line was maintained in MCDB 170 medium supplemented with 52 μg/ml BPE, 0.5 μg/ml hydrocortisone, 10 μg/ml hEGF, 5 μg/ml insulin, 50 μg/ml gentamicin, and 50 ng/ml amphotericin-B. The cell line was passaged every 2-3 days and maintained for no more than 8 population doublings (PD) from the PDᵢₙᵢₜᵢₐₗ.

Lipids

Oxidized LDL (protein concentration 2 mg/ml), Human LDL (protein concentration 5 mg/ml), Human HDL (protein concentration 10 mg/ml) were purchased from Biomedical Technologies Inc. Water-Soluble Cholesterol was obtained from Sigma (30 mg of pure cholesterol in 0.7 g total weight of solid, balanced in methyl-beta-cyclodextrin) and resuspended in sterile water.

In Vitro Incubation with Lipoproteins/Cholesterol

Two days prior to treatment/incubation, freshly passaged hTERT cells (1-3×10⁵ cells/2 mL) were plated in 100 mm tissue culture dishs (56 cm²/dish) with supplemented Mammary Epithelium Basal Medium (MEBM, Cambrex-Clonetech) to assure 60%-70% confluency on the day of treatment. Final concentration of lipoproteins used in culture was 20 μg/ml. Final concentration of Water-soluble Cholesterol was 2 and 4 μg/ml, since 10 and 20 μg/ml proved to be cytotoxic. At 48 hours post-lipid incubation, cells were treated with 33 ng/ml colcemid for 10 hours, harvested and scored for aneuploidy by metaphase chromosome analysis/karyotype.

Metaphase Chromosome Analysis

Following colcemid treatment, cells were harvested according to standard cytogenetic methods. The cells were hypotonicly treated in 750 mM KC1 for 15 minutes in a 37° C. water bath, fixed in 3:1 low-water methanol: acetic acid fixative for a minimum of 30 minutes on ice, following two further fixative washes and resuspension in fresh fix.

Cells were dropped onto wet glass slides (Fisherbrand Frosted Microscope Slides) and allowed to age overnight at room temperature. Metaphase spreads were stained with Giemsa (Gibco/Invitrogen Corporation) and the chromosomes counted. Genus 2.81® software was used for chromosome analysis (Applied Imaging). At least thirty metaphases were analyzed/scored per each sample.

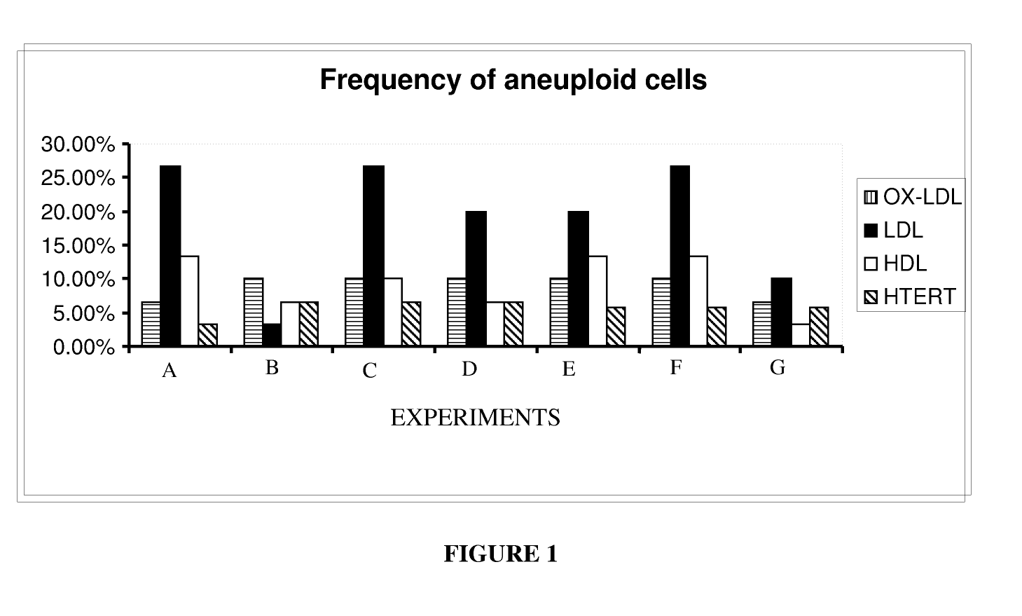

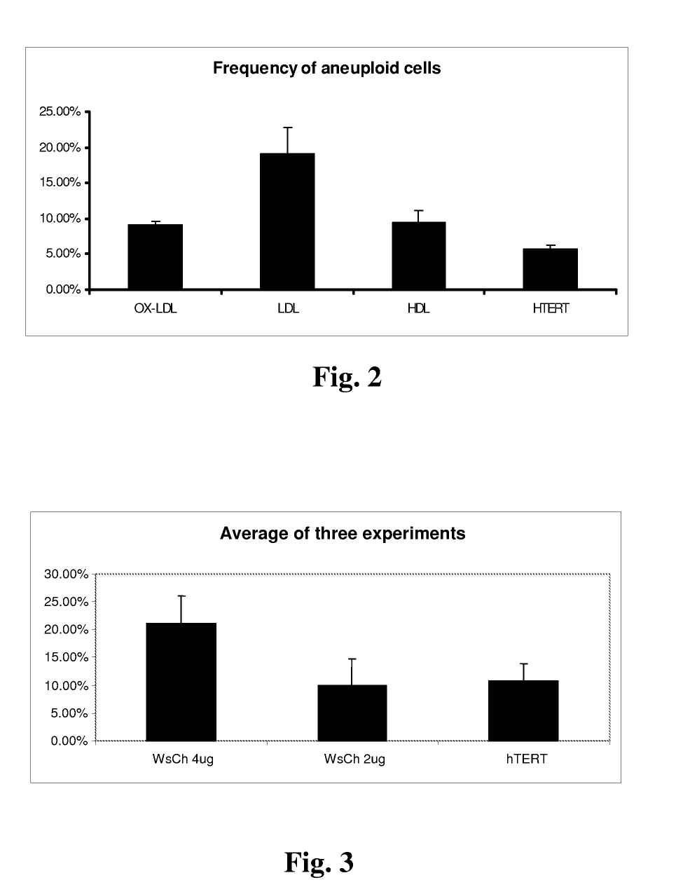

FIGS. 1 through 3 present the results from 7 experiments with LDL and HDL and 3 experiments with water soluble cholesterol. The data shows that cholesterol either alone or in the form of LDL promotes chromosome missegregation and the development of aneuploid cells, while HDL and oxidized LDL have a much smaller or no such aneugenic effect. Because cholesterol increases the rigidity of membranes, the increase in membrane rigidity leads to the aneugenic effect. Conversely, increasing membrane fluidity should counter the effect of cholesterol.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of identifying an agent capable of inhibiting chromosome missegregation comprising the steps of:
providing a test cell;
administering to said test cell an experimentally effective amount of a solution comprising a compound selected from the group consisting of cholesterol and low-density-lipoprotein;
exposing the tests cell to an agent suspected of inhibiting chromosome missegregation;
allowing the test cell to reproduce, thereby producing a population of progeny cells; and
determining the number of aneuploid cells in said population of progeny cells, wherein a low frequency of aneuploid cells in said population is indicative that the agent is capable of inhibiting chromosome missegregation.

2. The method of claim 1 where the test cell permanently expresses telomerase reverse transcriptase.

3. The method of claim 1 where the low-density-lipoprotein is Human low-density-lipoprotein.

4. The method of claim 1 where the concentration of low-density-lipoprotein is about 20 μg/ml.

5. The method of claim 1 where the cholesterol is water-soluble.

6. The method of claim 5 where the concentration of water-soluble cholesterol is between about 2 and 4 μg/ml.

7. The method of claim 1 where the number of anomalous cells in said population of progeny cells is determined using standard metaphase chromosome analysis.

8. A method of identifying an agent capable of inhibiting chromosome missegregation comprising the steps of:
providing a test cell that expresses telomerase reverse transcriptase;
administering to said test cell an experimentally effective amount of a compound selected from the group consisting of cholesterol and low-density-lipoprotein;
exposing the tests cell to an agent suspected of inhibiting chromosome missegregation;
allowing the test cell to reproduce, thereby producing a population of progeny cells; and
determining the number of aneuploid cells in said population of progeny cells, wherein a low frequency of aneuploid cells in said population is indicative that the agent is capable of inhibiting chromosome missegregation.

9. A method of identifying an agent capable of inhibiting chromosome missegregation comprising the steps of:
providing a test cell;
administering to said test cell a solution having a final concentration of between about 2 and 4 μg/ml cholesterol;
exposing the test cell to an agent suspected of inhibiting chromosome missegregation;
allowing the test cell to reproduce, thereby producing a population of progeny cells; and
determining the number of anomalous cells in said population of progeny cells, wherein said anomalous cells are selected from the group consisting of aneuploid cells, cells comprising a chromosome having a break, comprising a chromosome having a translocation, wherein a low frequency of anomalous cells in said population is indicative that the agent is capable of inhibiting chromosome missegregation.

10. The method of claim 9 where the test cell expresses telomerase reverse transcriptase.

11. A method of identifying an agent capable of inhibiting chromosome missegregation comprising the steps of:
providing a test cell;
administering to said test cell a solution having a final concentration of about 20 μg/ml lipoprotein;
exposing the test cell to an agent suspected of inhibiting chromosome missegregation;
allowing the test cell to reproduce, thereby producing a population of progeny cells; and
determining the number of aneuploid cells in said population of progeny cells, wherein a low frequency of aneuploid cells in said population is indicative that the agent is capable of inhibiting chromosome missegregation.

12. The method of claim 11 where the test cell expresses telomerase reverse transcriptase.

13. A method of identifying an agent capable of modulating membrane fluidity comprising the steps of:
providing a test cell;
administering to said test cell an experimentally effective amount of a solution comprising a compound selected from the group consisting of cholesterol and low-density-lipoprotein;
exposing the tests cell to an agent suspected of inhibiting chromosome missegregation;
allowing the test cell to reproduce, thereby producing a population of progeny cells; and
determining the number of aneuploid cells in said population of progeny cells, wherein a low frequency of aneuploid cells in said population is indicative that the agent is capable of inhibiting chromosome missegregation.

14. The method of claim 13 where the test cell permanently expresses telomerase reverse transcriptase.

15. The method of claim 13 where the low-density-lipoprotein is Human low-density-lipoprotein.

16. The method of claim 13 where the concentration of low-density-lipoprotein is about 20 μg/ml.

17. The method of claim 13 where the cholesterol is water-soluble.

18. The method of claim 17 where the concentration of water-soluble cholesterol is between about 2 and 4 μg/ml.

19. The method of claim 13 where the number of anomalous cells in said population of progeny cells is determined using standard metaphase chromosome analysis.

20. A method of identifying an agent capable of modulating membrane fluidity comprising the steps of:
providing a test cell that expresses telomerase reverse transcriptase;
administering to said test cell an experimentally effective amount of a compound selected from the group consisting of cholesterol and low-density-lipoprotein;
exposing the tests cell to an agent suspected of inhibiting chromosome missegregation;
allowing the test cell to reproduce, thereby producing a population of progeny cells; and
determining the number of aneuploid cells in said population of progeny cells, wherein a low frequency of aneuploid cells in said population is indicative that the agent is capable of inhibiting chromosome missegregation.

21. A method of identifying an agent capable of modulating membrane fluidity comprising the steps of:
providing a test cell;
administering to said test cell a solution having a final concentration of between about 2 and 4 μg/ml cholesterol;
exposing the test cell to an agent suspected of inhibiting chromosome missegregation;
allowing the test cell to reproduce, thereby producing a population of progeny cells; and
determining the number of anomalous cells in said population of progeny cells, wherein said anomalous cells are selected from the group consisting of aneuploid cells, cells comprising a chromosome having a break, comprising a chromosome having a translocation, wherein a low frequency of anomalous cells in said population is indicative that the agent is capable of inhibiting chromosome missegregation.

22. The method of claim 21 where the test cell expresses telomerase reverse transcriptase.

23. A method of identifying an agent capable of modulating membrane fluidity comprising the steps of:
providing a test cell;
administering to said test cell a solution having a final concentration of about 20 μg/ml lipoprotein;
exposing the test cell to an agent suspected of inhibiting chromosome missegregation;
allowing the test cell to reproduce, thereby producing a population of progeny cells; and
determining the number of aneuploid cells in said population of progeny cells, wherein a low frequency of aneuploid cells in said population is indicative that the agent is capable of inhibiting chromosome missegregation.

24. The method of claim 23 where the test cell expresses telomerase reverse transcriptase.
